(19) United States
(12) Patent Application Publication
(10) Pub. No.: US 2016/0128929 A1
(43) Pub. Date: May 12, 2016

(54) METHOD FOR PRODUCTION OF STRUCTURED LIQUID COMPOSITIONS AND STRUCTURED LIQUID COMPOSITIONS

(71) Applicant: Conopco, Inc., d/b/a UNILEVER, Englewood Cliffs, NJ (US)

(72) Inventors: Petrus Martinus Maria BONGERS, (US); Michael John EGAN, Liverpool (GB); Graeme Neil IRVING, Bebington, Wirral (GB); Sally WOOD, Wirral (GB)

(21) Appl. No.: 14/896,517

(22) PCT Filed: May 19, 2014
(86) PCT No.: PCT/EP2014/060220

§ 371 Date: Dec. 7, 2015

(30) Foreign Application Priority Data
Jun. 14, 2013 (EP) 13172073.2

(51) Int. Cl. A61K 8/92; A61K 8/34; A61Q 15/00; A61K 8/37; A61K 8/28; A61K 8/26

(52) U.S. Cl. CPC A61K 8/92; A61K 8/28; A61K 8/26; A61Q 15/00; A61K 8/37; A61K 8/342; A61K 2800/10

(57) ABSTRACT

The present invention relates to personal care compositions containing non-ionic surfactants and fatty compounds, which have a structured composition to provide rheological properties to these compositions. The compositions can be prepared by applying a Controlled Deformation Dynamic Mixer. By using this mixer, compositions having a relatively high viscosity can be prepared, while the concentration of active compounds is relatively low.

### FIELD OF THE INVENTION

The present invention relates to a method for the production of a structured liquid composition that can be used as a personal care composition, for example as deodorant or antiperspirant, by using a Controlled Deformation Dynamic Mixer. The present invention also relates to a structured liquid composition containing fatty compound, non-ionic surfactant, and water, and that has a high viscosity with a small amount of these compounds.

### BACKGROUND TO THE INVENTION

Mixing can be described as either distributive or dispersive. In a multi-phase material comprising discrete domains of each phase, distributive mixing seeks to change the relative spatial positions of the domains of each phase, whereas dispersive mixing seeks to overcome cohesive forces to alter the size and size distribution of the domains of each phase. Most mixers employ a combination of distributive and dispersive mixing although, depending on the intended application the balance will alter. For example a machine for mixing peanuts and raisins will be wholly distributive so as not to damage the things being mixed, whereas a blender/homogeniser will be dispersive.

EP 194 812 A2 and WO 96/20270 describe a cavity transfer mixer (CTM). WO 96/20270 also describes a ‘Controlled Deformation Dynamic Mixer’ (CDDM). This type of mixer has stator and rotor elements with opposed cavities which, as the mixer operates, move past each other across the direction of bulk flow through the mixer. The CDDM distinguishes from the CTM in that material is also subjected to extensional deformation. The extensional flow and efficient dispersive mixing is secured by having confronting surfaces with cavities arranged such that the cross sectional area for bulk flow of the liquid through the mixer successively increases and decreases by a factor of at least 5 through the apparatus. The CDDM combines the distributive mixing performance of the CTM with dispersive mixing performance. Also WO 2012/089474 A1 describes a CTM and a CDDM. WO 96/20270 further describes that this type of mixer can be used for the production of structured liquids, such as compositions containing surfactants (anionic, cationic, non-ionic, zwitterionic). The production of a fabric conditioning composition is described, and the viscosity of the produced compositions ranges from 40 to 155 mPa·s.

Personal care compositions mostly refer to compositions intended for topical application to the skin or hair. Many of such compositions are in the form of a structured liquid. Generally this means that the liquid is a stable dispersion whose in-use properties are a function of microstructure (structure at a microscopic scale). Structured liquids cannot be simply described by their composition, and their properties are a function of how they have been processed. For example, although some proportion of materials may be dissolved in one or more of the liquid ingredients, another fraction may be dispersed throughout the volume in droplets or particles within a range of sizes. Microstructure is generally characterised using microscopy (light or electron microscopy), and the rheology of structured liquid systems is usually determined and compared. In many cases the degree of dispersion of particles or droplets is determined using optical light microscopy or scanning electron microscopy.

Many personal care compositions are a mixture of surfactants (e.g. non-ionic, anionic, cationic, and/or zwitter-ionic), neutral fatty compounds (e.g. triglycerides, fatty alcohols, waxes), and water. These compounds may form a microstructure in the form of a structured liquid, determined by their preparation method. For example the surfactants may be organised in micelles, within a continuous aqueous phase. Or the surfactants form planar sheets, wherein the hydrophilic heads are at two outsides of these planar sheets and hydrophobic tails are at the inside, therewith forming a lamellar structure of these sheets with water in between the sheets (see e.g. J. Eastoe, Surfactant Aggregation and Adsorption at Interfaces, ch. 4 in: T. Cosgrove (ed.), Colloid Science; Principles, Methods and Applications; Blackwell Publishing Ltd., Oxford (UK), 2005).

US 2010/0143280 discloses a method for preparing a personal care composition, comprising a surfactant and a fatty compound, including a mixing step conducted by using a homogeniser having a rotating member.

US 2007/0027050 A1 discloses a liquid crystalline structured cleansing and moisturising composition, having a broad viscosity range, and containing the anionic surfactant C6 to C16 alkyl mono sulfosuccinate(s) and polyols like the polyethylene glycols.

WO 03/074020 A1 discloses an ordered liquid crystalline structured cleansing composition containing an anionic surfactant and organogel particles that generally comprise a vegetable oil and a waxy compound.

WO 2005/063174 A1 discloses an ordered liquid crystalline structured cleansing composition containing an anionic surfactant and an amphoteric surfactant,

US 2011/0300093 A1 discloses cosmetic compositions containing various surfactants and a polymer, however no fatty compound.

### SUMMARY OF THE INVENTION

Many of the processes for preparation of structured liquids do not manage to yield compositions which have consistent and repeatable rheological properties. Therefore manufacturers require improved processes which can be used to consistently produce structured liquids. Moreover the manufacturers wish to improve production methods and products, for example by decrease of energy consumption, or decrease of the concentration of active compounds in the formulation, while keeping the performance of the product at least as good as the standard product. This way resources (raw materials, energy) can be saved, additionally leading to cheaper products. Also nowadays consumers demand more and more products which consume less energy and resources upon production, transport and use.

Therefore there is a desire to provide personal care products that can be prepared and used with less valuable resources than common processes and products. Therefore it is an object of the invention to provide a method for the production of a structured liquid (that can be used as a personal product, e.g. a cream, a deodorant and/or an antiperspirant), that leads to more efficient use of raw materials, to reduction of the amount of raw materials needed, while keeping the same functionality of the structured liquids. It is another object of the invention to provide a process that can be used to consistently produce personal care compositions of the same quality and structure as common products. Another object of the invention is to provide a composition that does not require a large amount or high concentration of ingredients, and that nevertheless have the right consistency and viscosity to be functional as personal care composition.

We have now determined that this objective can be met by a method for preparation of a structured liquid, that contains water, fatty compound and one or more non-ionic surfactants and that can be used as a personal care composition, e.g. a skin cream and/or deodorant and/or an antiperspirant. The non-ionic surfactants are mild to the skin. The method uses a Controlled Deformation Dynamic Mixer type. By this method structured liquids for use as personal care composition can be produced that do not require high concentrations of actives, and still have a good consistency and viscosity to be functional as personal care composition, e.g. as skin cream and/or deodorant and/or antiperspirant. The objective is also met by a structured liquid composition, having a relatively low concentration of fatty compound and non-ionic surfactant, while still having a dynamic viscosity which is similar to compositions having a higher content of fatty compound and surfactant. By this increase of viscosity, the concentration of raw materials can be decreased, while the functionality of the formulation is kept the same as if with a higher raw material concentration.

Accordingly in a first aspect the invention provides a method for production of a structured liquid composition comprising water, a fatty compound having a melting point of at least 25° C. at a concentration of at least 1% by weight, and one or more non-ionic surfactants at a concentration of at least 1% by weight, comprising the step:

a) mixing the fatty compound in liquid form with a mixture containing the one or more non-ionic surfactants in liquid form and water, or mixing the fatty compound in liquid form with the one or more non-ionic surfactants in liquid form, and mixing this mixture with water;
characterised in that in a next step
b) the mixture from step a) is introduced into a distributive and dispersive mixing apparatus of the Controlled Deformation Dynamic Mixer type,
wherein the mixer is suitable for inducing extensional flow in a liquid composition,
and wherein the mixer comprises closely spaced confronting surfaces at least one having a series of cavities therein in which the cavities on each surface are arranged such that, in use, the cross-sectional area for flow of the liquid successively increases and decreases by a factor of at least 5 through the apparatus.

In a second aspect the present invention provides a structured liquid obtainable by the method according to the invention.

The second aspect of the invention also provides a structured liquid composition comprising water, and one or more fatty compounds having a melting point of at least 25° C. at a concentration ranging from 1% to 4% by weight, and

one or more non-ionic surfactants at a concentration ranging from 1% to 8% by weight, and water,
and wherein the total concentration of anionic surfactants, cationic surfactants, and zwitterionic surfactants is maximally 3% by weight,
and wherein the structured liquid has a dynamic viscosity of at least 80,000 mPa·s, preferably at least 100,000 mPa·s, measured using a Brookfield RV viscometer, fitted with a T-bar T-E spindle, at a rotational speed of 5 rpm, and a temperature of 25° C.

The second aspect of the invention also provides a structured liquid composition comprising water, and one or more fatty compounds having a melting point of at least 25° C. at a concentration ranging from 2% to 5% by weight, and

one or more non-ionic surfactants at a concentration ranging from 4% to 8% by weight, and water,
and wherein the total concentration of anionic surfactants, cationic surfactants, and zwitterionic surfactants is maximally 3% by weight,
and wherein the structured liquid has a dynamic viscosity of at least 60,000 mPa·s, preferably at least 80,000 mPa·s, measured using a Brookfield RV viscometer, fitted with a T-Bar T-D spindle at a rotational speed of 10 rpm, and a temperature of 25° C.

In a third aspect the present invention provides use of a structured liquid, prepared according to the method of first aspect of the invention and comprising an antiperspirant active, preferably comprising an aluminium compound and/or a zirconium compound, or according to the second aspect of the invention as deodorant or antiperspirant.

### DESCRIPTION OF FIGURES

FIG. 1: Schematic representation of a Cavity Transfer Mixer (CTM); 1: stator, 2: annulus; 3: rotor; with cross-sectional views below.

FIG. 2: Schematic representation of a Controlled Deformation Dynamic Mixer (CDDM); 1: stator, 2: annulus; 3: rotor; with cross-sectional views below.

FIG. 3: Schematic representation of a preferred embodiment of the CDDM apparatus, cross-sectional view (direction of bulk flow preferably from left to right).

FIG. 4: Schematic representation of a preferred embodiment of the CDDM apparatus, cross-sectional view (direction of bulk flow preferably from left to right).

FIG. 5: Dynamic viscosity (in mPa·s) as function of the concentration of active materials in the compositions (100% has formulation as in Table 2, and diluted samples), from example 1; linear trendlines indicated. Measured using Brookfield viscometer, T-E Spindle, 10 rpm, 25° C., measurement 1 minute after initiating the measurement procedure.

:control samples (did not pass CDDM), ▴ samples that passed CDDM at 20 mL/s and 10,000 rpm; *: samples that passed static CDDM at 20 mL/s.

FIG. 6: The yield stress as function of the concentration of active materials in the compositions (100% has formulation as in Table 2, and diluted samples), from example 1.

: control samples (did not pass CDDM), ▴ samples that passed CDDM at 80 mL/s and 10,000 rpm; *: samples that passed static CDDM at 80 mL/s.

FIG. 7: Dynamic viscosity (in mPa·s) as function of the concentration of active materials in the compositions (100% has formulation as in Table 4, and diluted samples), from example 2; linear trendlines indicated. Measured using Brookfield viscometer, T-bar T-D Spindle, 10 rpm, 25° C., measurement 1 minute after initiating the measurement procedure.

: control samples (did not pass CDDM), ▴ samples that passed CDDM at 80 mL/s and 10,000 rpm; *: samples that passed static CDDM at 80 mL/s.

FIG. 8: Yield stress (in Pa) as function of the concentration of active materials in the compositions (100% has formulation as in Table 4, and diluted samples), from example 2; linear trendlines indicated.

: control samples (did not pass CDDM), ▴ samples that passed CDDM at 80 mL/s and 10,000 rpm; *: samples that passed static CDDM at 80 mL/s.

FIG. 9: Dynamic viscosity (in mPa·s) as function of the concentration of active materials in the compositions (100% has formulation as in Table 6, and diluted samples), from example 3; linear trendlines indicated. Measured using Brookfield viscometer, T-E Spindle, 5 rpm, 25° C., measurement 1 minute after initiating the measurement procedure.

: control samples (did not pass CDDM), ▴ samples that passed CDDM at 80 mL/s and 10,000 rpm; *: samples that passed static CDDM at 80 mL/s (these ‘static samples’ are average of two measurements).

FIG. 10: Yield stress (in Pa) as function of the concentration of active materials in the compositions (100% has formulation as in Table 6, and diluted samples), from example 3; linear trendlines indicated.

: control samples (did not pass CDDM), ▴ samples that passed CDDM at 80 mL/s and 10,000 rpm; *: samples that passed static CDDM at 80 mL/s.

### DETAILED DESCRIPTION OF THE INVENTION

Unless defined otherwise, all technical and scientific terms used herein have the same meaning as commonly understood by one of ordinary skill in the art. All percentages, unless otherwise stated, refer to the percentage by weight. The abbreviation ‘wt %’ refers to percentage by weight. In case a range is given, the given range includes the mentioned endpoints. Ambient or room temperature is considered to be a temperature between about 15° C. and about 25° C., preferably between 17° C. and 24° C., preferably between 20° C. and 23° C.

### Cavity Transfer Mixers (CTMs) and Controlled Deformation Dynamic Mixers (CDDMs)

Similar as in WO 96/20270, CTMs are defined as mixers comprising confronting surfaces, at least one of the surfaces, preferably both surfaces, having a series of cavities formed therein in which the surfaces move relatively to each other and in which a liquid material is passed between the surfaces and flows along a pathway successively through the cavities in each surface. Generally the cavities are arranged such that the cross sectional area for flow of the liquid successively increases and decreases by a factor of about 3 through the apparatus. For further description of the CTM we refer to WO 96/20270 and WO 2012/089474 A1, which are herein incorporated by reference.

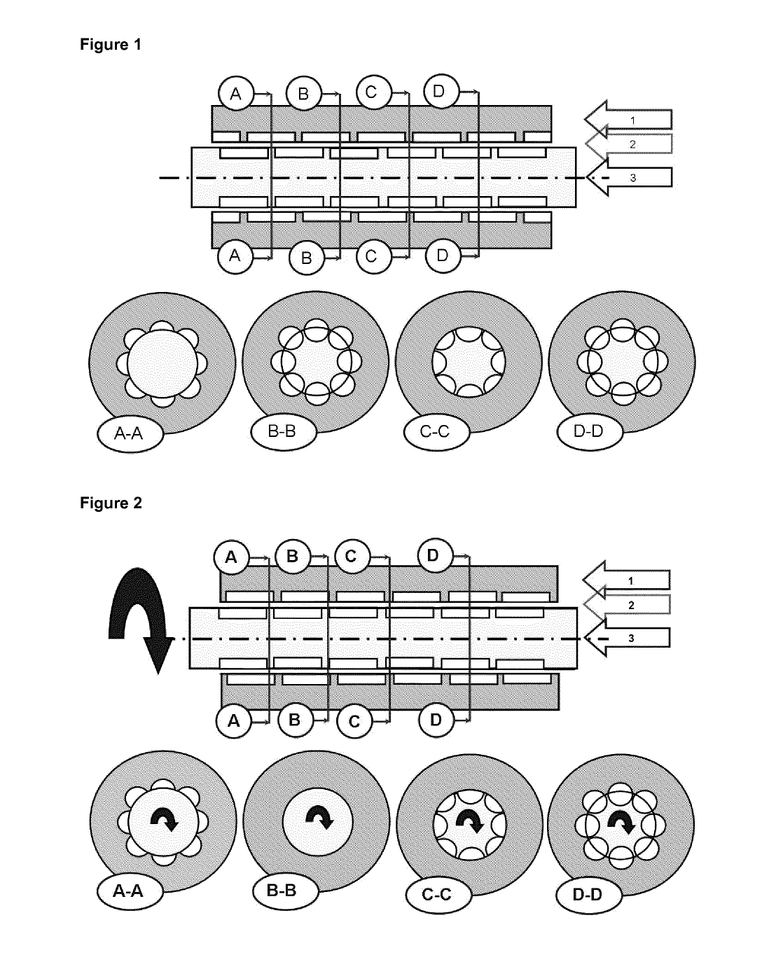

CTMs are exemplified by reference to FIG. 1 which displays an axial section and four transverse radial sections through a CTM configured as a ‘concentric cylinder’ device and comprising an inner rotor journalled within an outer stator. Briefly, the axial section shows the relative axial positions of rotor and stator cavities which are time invariant, whereas the transverse sections (A-A, B-B, C-C, D-D) demonstrate the axial variation in the available cross-sectional area for material flow axially: The key feature to note is that there is little variation in the cross-sectional area for flow as the material passes axially down the device. Also the CDDM is described in WO 96/20270 and WO 2012/089474 A1. CDDMs are distinguished from CTMs by their description as mixers: in addition to shear, significant extensional flow and efficient distributive and dispersive mixing may be secured by providing an apparatus having confronting surfaces and cavities therein in which the cavities are arranged such that the cross sectional area for flow of the liquid successively increases and decreases by a factor of at least 5 through the apparatus.

CDDMs are exemplified by reference to FIG. 2 which displays an axial section and four transverse radial sections through a CDDM configured as a ‘concentric cylinder’ device comprising an inner rotor journalled within an outer stator. Briefly, the axial section shows the relative axial positions of rotor and stator cavities which are time invariant, whereas the transverse sections (A-A, B-B, C-C, D-D) demonstrate the axial variation in the available cross-sectional area for material flow axially: Clearly there is a significant variation in the cross-sectional area for flow as the material passes axially through the annulus formed between the ‘rotor rings’ and the ‘stator rings’ (B-B), and between confronting rotor cavities and stator cavities (D-D).

By comparison of FIG. 1 and FIG. 2, CDDMs are distinguished from CTMs by the relative position of the rotor and stator and consequent incorporation of an extensional component of flow. Hence CDDMs combine the distributive mixing performance of CTMs with the dispersive mixing performance of multiple expansion-contraction static mixers.

Although the CDDM generally has a moving part, it may also be run in static mode, meaning that one or more fluids are pumped through the apparatus without rotation of the rotor. This is called the static mode of the apparatus.

### Method for Production of Structured Liquid

In a first aspect the invention provides a method for production of a structured liquid composition comprising water, a fatty compound having a melting point of at least 25° C. at a concentration of at least 1% by weight, and one or more non-ionic surfactants at a concentration of at least 1% by weight, comprising the step:

a) mixing the fatty compound in liquid form with a mixture containing the one or more non-ionic surfactants in liquid form and water, or
mixing the fatty compound in liquid form with the one or more non-ionic surfactants in liquid form, and mixing this mixture with water;
characterised in that in a next step
b) the mixture from step a) is introduced into a distributive and dispersive mixing apparatus of the Controlled Deformation Dynamic Mixer type,
wherein the mixer is suitable for inducing extensional flow in a liquid composition,
and wherein the mixer comprises closely spaced confronting surfaces at least one having a series of cavities therein in which the cavities on each surface are arranged such that, in use, the cross-sectional area for flow of the liquid successively increases and decreases by a factor of at least 5 through the apparatus.

In the context of the present invention, the materials are defined in the following way.

A ‘personal care composition’ refers to compositions intended for topical application to the skin or hair. They may be used as rinse-off formulations, wherein the composition is rinsed off with water (e.g. shampoo, or body wash), or may be leave-on formulations (e.g. deo cream, or skin cream). The personal care compositions may be in the form of liquid, semi-liquid, cream, lotion or gel compositions. Examples of personal care compositions include but are not limited to shampoo, conditioning shampoo, hair conditioner, body wash, moisturising body wash, shower gels, skin cleansers, cleansing milks, hair and body wash, in shower body moisturizer, shaving preparations, skin creams, skin lotions, and deo creams.

A ‘structured liquid’ refers to a composition that is structured by its microstructure, as explained herein before. The structured liquid has a rheology that confers stability on the personal care composition. Stability means that the composition keeps its structure during normal shelf life, during at least 6 months, preferably at least 12 months at ambient temperature. The term ‘structured liquid’ may relate to a liquid, semi-liquid, cream, or lotion.

A ‘surfactant’ is a compound having a hydrophilic head and a hydrophobic tail, and that can be used to stabilise mixtures of hydrophilic and hydrophobic compounds which without surfactant would not mix. Generally surfactants may be non-ionic, anionic, cationic, or zwitterionic.

A ‘fatty compound’ is defined as a neutral compound under neutral pH conditions that is non-volatile at normal conditions (room temperature, atmospheric pressure), water-insoluble, non-silicone, and does not mix with water without stabiliser like a surfactant. By ‘water-insoluble’ is meant that the maximum solubility in water is 0.1% by weight, at 25° C. In the context of the present invention, surfactants are not considered to be fatty compounds.

In step a) of the method of the invention a premix is made of the ingredients of the composition. In one possible way to make the premix, the fatty compound in liquid form is mixed with a mixture containing the one or more non-ionic surfactants in liquid form and water.

The fatty compound is solid or semi-solid at room temperature, and the fatty compound is melted at a temperature higher than the melting point of the fatty compound, preferably at a temperature of at least 60° C., preferably at least 70° C. The maximum melting temperature in this step preferably is 110° C., preferably maximally 100° C. Preferably the temperature ranges from 60 to 80° C.

Preferably the non-ionic surfactant is solid or semi-solid at room temperature. In that case, preferably, the non-ionic surfactant is melted at a temperature higher than the melting point of the non-ionic surfactant, preferably at a temperature of at least 60° C., preferably at least 70° C. Preferably the temperature ranges from 60 to 80° C.

The non-ionic surfactant is dispersed in at least part of the water of the total formulation. Dispersing the non-ionic surfactant may be performed at relatively low temperature, after which the temperature of the mixture is increased, in order to melt the non-ionic surfactant. The dispersing of non-ionic surfactant may also be done when the non-ionic surfactant and water have been brought to an elevated temperature separately already. The temperature at which the dispersion of non-ionic surfactant in water is brought into contact with the fatty compound is similar to the temperature of the fatty compound in liquid form. Hence preferably the temperature of the aqueous phase with non-ionic surfactant is at least 60° C., preferably at least 70° C., and preferably lower than 110° C. Preferably, the temperature of the mixing in step a) ranges from 60° C. to 80° C.

The two phases (molten fatty compound and mixture of non-ionic surfactants and water) are preferably mixed in a vessel under high shear, for example by using a Silverson high shear mixer (ex Silverson Machines Ltd., Chesham, Buckinghamshire, UK), operated at a rotational speed of preferably 3,000-5,000 rpm, which preferably generates a shear rate of 40,000-50,000 s⁻¹.

Alternatively, the mixtures in step a) of the method of the invention are gently mixed with low shear processes, and not with a high shear operation.

The mixture may be further diluted with water, which preferably is at a low temperature, for example about 5-50° C., preferably at about 20-40° C. The optional dilution water may also be at a similar temperature as the mixture in step a). The optional dilution water may contain other ingredients of the compositions like preservative, fragrance, and antiperspirant active compound. The optional water and optional further ingredients are then gently mixed into the composition of step a).

Preferably, optional ingredients like preservative, fragrance, and antiperspirant active compound are added to the composition from step a) after the temperature of the mixture has decreased to a temperature of 65° C. or lower, preferably to a temperature of 60° C. or lower. Preferably optional ingredients are added at a temperature ranging from 40 to 50° C. These optional ingredients may be gently mixed into the composition, or may be mixed with the composition under high shear.

Subsequently this mixture is brought into the CDDM mixer, to perform mixing step b). This mixing may be done at a temperature ranging from 5° C. to 110° C. In case the mixture prepared in step a) is mixed under high shear, then preferably the mixing step b) is performed at a temperature ranging from 5° C. to 30° C., preferably from 15° C. to 25° C. In case the mixture prepared in step a) is mixed under low shear, then preferably the mixing step b) is performed at a temperature higher than the melting points of the fatty compounds and the non-ionic surfactants. Preferably the temperature is then at least 60° C., preferably at least 70° C. The maximum temperature in this step preferably is 110° C., preferably maximally 100° C. Preferably the temperature ranges from 60 to 80° C.

Summarising, this process can be described by the following steps:
1. preparing a mixture of non-ionic surfactant and water;
2. mixing molten fatty compound into this mixture, preferably using a low shear mixing operation;
3. optionally adding water and/or further ingredients;
4. mixing this mixture in CDDM apparatus.

These steps 1, 2, and 3 together form step a) of the method of the invention, and this step 4 forms step b) of the method of the invention.

Optionally, further water is added to the mixture from step a) prior to step b), and/or to the mixture obtained from step b).

Alternatively, the premix in step a) is made by mixing the fatty compound in liquid form with the one or more non-ionic surfactants in liquid form, and mixing this mixture with water. The fatty compound is solid or semi-solid at room temperature. Preferably the non-ionic surfactant is solid or semi-solid at room temperature. Preferably the fatty compound and non-ionic surfactant are melted at a temperature of at least 60° C., preferably at least 70° C. The maximum melting temperature in this step preferably is 110° C., preferably maximally 100° C. Preferably the temperature ranges from 60 to 80° C. Preferably the two materials are mixed using a high-shear mixer.

This mixture is mixed with water, which preferably is at a similar temperature as the mixture of fatty compound and non-ionic surfactants The two phases (mixture of molten fatty compound and non-ionic surfactants and water) are preferably mixed in a vessel under high shear, for example by using a Silverson high shear mixer (ex Silverson Machines Ltd., Chesham, Buckinghamshire, UK), operated at a rotational speed of preferably 3,000-5,000 rpm, which preferably generates a shear rate of 40,000-50,000 s⁻¹.

Alternatively, the mixtures in step a) of the method of the invention are gently mixed with low shear processes, and not with a high shear operation.

The mixture may be further diluted with water, which preferably is at a low temperature, for example about 5-50° C., preferably at about 20-40° C. The optional dilution water may also be at a similar temperature as the mixture in step a). The optional dilution water may contain other ingredients of the compositions like preservative, fragrance, and antiperspirant active compound. The optional water and optional further ingredients are then gently mixed into the composition of step a).

Subsequently this mixture is brought into the CDDM mixer, to perform mixing step b). This mixing may be done at a temperature ranging from 5° C. to 110° C. In case the mixture prepared in step a) is mixed under high shear, then preferably the mixing step b) is performed at a temperature ranging from 5° C. to 30° C., preferably from 15° C. to 25° C. In case the mixture prepared in step a) is mixed under low shear, then preferably the mixing step b) is performed at a temperature higher than the melting points of the fatty compounds and the non-ionic surfactants. Preferably the temperature is then at least 60° C., preferably at least 70° C. The maximum temperature in this step preferably is 110° C., preferably maximally 100° C. Preferably the temperature ranges from 60 to 80° C.

Summarising, this process can be described by the following steps:
1. preparing a mixture of non-ionic surfactant and fatty compound;
2. mixing of water with this mixture, preferably using a low shear mixing operation;
3. optionally adding water and further ingredients, and mixing this in a low shear mixing operation;
4. mixing this mixture in CDDM apparatus.

These steps 1, 2, and 3 together form step a) of the method of the invention, and this step 4 forms step b) of the method of the invention.

Optionally, further water is added to the mixture from step a) prior to step b), and/or to the mixture obtained from step b).

### Non-Ionic Surfactants

The compositions prepared according to the method of the invention comprise one or more non-ionic surfactants at a concentration of at least 1% by weight of the final composition.

Non-ionic surfactants have the advantage that they are generally milder to the skin than some other surfactants, e.g. anionic surfactants. Generally, a non-ionic surfactant has a HLB-value of at least 1. Preferably the concentration of non-ionic surfactants ranges from 1% to 8% by weight of the final composition.

In one preferred embodiment the concentration of non-ionic surfactants ranges from 1% to 8% by weight, preferably from 1% to 6% by weight, preferably from 1.5% to 4% by weight.

In another preferred embodiment the concentration of non-ionic surfactants ranges from 4% to 8% by weight, preferably from 4% to 7% by weight.

Preferably the one or more non-ionic surfactants have a weighted average HLB value ranging from 3 to 12, preferably ranging from 4 to 10, preferably from 4 to 8. This preferred HLB value of the one or more non-ionic surfactants can be achieved by a single type of non-ionic surfactant, or a combination of at least two types of non-ionic surfactants. More preferred, in case a combination of non-ionic surfactants is used, the non-ionic surfactants comprise a non-ionic surfactant having a HLB value ranging from 2 to 6.5, preferably from 4 to 6, and a non-ionic surfactant having a HLB value ranging from 6.5 to 18, preferably from 12 to 18. The average HLB value of such a combination of non-ionic emulsifiers can be calculated by the weight average HLB value of the constituents.

Preferably, the one or more non-ionic surfactants have a melting point of at least 25° C., preferably 30° C. or higher, preferably 40° C. or higher, in view of stability of the composition obtained by the method of the invention. Preferably, such melting point is up to about 90° C., more preferably up to about 80° C., still more preferably up to about 70° C., even more preferably up to about 65° C.

A preferred range of non-ionic surfactants comprises a hydrophilic moiety provided by a polyalkylene oxide (polyglycol), and a hydrophobic moiety provided by an aliphatic hydrocarbon, preferably containing at least 10 carbons and commonly linear. The hydrophobic and hydrophilic moieties can be linked via an ester or ether linkage, possibly via an intermediate polyol such as glycerol. A preferred range of emulsifiers comprises polyethylene glycol ethers.

Preferably, the polyalkylene oxide is often selected from polyethylene oxide and polypropylene oxide or a copolymer of ethylene oxide and especially comprises a polyethylene oxide. The number of alkylene oxide and especially of ethoxylate units within suitable emulsifiers is preferably selected within the range of from 2 to 100. Emulsifiers with a mean number of ethoxylate units in the region of 2 can provide a lower HLB value of below 6.5 (depending on the specific hydrophobic tail) and those having at least 4 such units provide a higher HLB value of above 6.5 and especially those containing at least 10 ethoxylate units which provide an HLB value of above 10.

Preferably, if a non-ionic surfactant having a HLB value ranging from 6.5 to 18 is present, then that non-ionic surfactant comprises one or more polyethylene glycol alkyl ethers, wherein the alkyl moiety preferably comprises hexadecyl or octadecyl, and/or wherein the polyethylene glycol preferably has a degree of alkoxylation ranging from 4 to 30, preferably from 4 to 20. A preferred non-ionic surfactant is Steareth-20, which is a polyethylene glycol (n=20) octadecyl ether, (also called PEG-20 stearate), HLB-value of about 15.3, melting point 44-46° C., with the following structure:

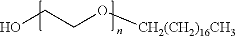

Preferably, if a non-ionic surfactant having a HLB value ranging from 2 to 6.5 is present, then that non-ionic surfactant comprises a glyceryl mono-ester of fatty acids having from 16 to 18 carbon atoms. Such a non-ionic surfactant is also generally known as a monoglyceride. Another preferred non-ionic surfactant having a HLB value ranging from 2 to 6.5 is Steareth-2 (also called PEG-20 stearate), which is a diethylene glycol octadecyl ether (with n=2 in the structure above), HLB-value of about 4.9, melting point 44-45° C., with the following structure:

Preferably, if a non-ionic surfactant having a HLB value ranging from 6.5 to 18 and a non-ionic surfactant having a HLB value ranging from 2 to 6.5 are present, then the ratio between the high HLB non-ionic surfactants and the low HLB non-ionic surfactants ranges from 1:15 to 1:1 (high HLB:low HLB), preferably from 1:12 to 1:1, preferably from 1:8 to 1:1.5, preferably from 1:8 to 1:3.

The total concentration of non-ionic surfactants in the composition made according to the method of the invention ranges from 1% to 8% by weight of the composition.

In case the composition is in the form of a cream (meaning a composition which is considered by users to be relatively thick), then preferably the concentration of non-ionic surfactants ranges from 4% to 8% by weight of the composition, more preferred from 4% to 7% by weight of the composition. In such case (the composition in the form of a cream) preferably the non-ionic surfactants comprise a non-ionic surfactant having a HLB value ranging from 2 to 6.5, preferably from 4 to 6, at a concentration ranging from 3% to 7%, preferably from 4% to 6% by weight, and/or a non-ionic surfactant having a HLB value ranging from 6.5 to 18, preferably from 12 to 18, at a concentration ranging from 0.5% to 3%, preferably from 1% to 2.5% by weight.

In case the composition is in the form of a lotion (meaning a composition which is considered by users to be relatively thin), then preferably the concentration of non-ionic surfactants ranges from 1% to 8% by weight of the composition. Preferably the concentration ranges from 1% to 6% by weight, preferably from 1% to 4% by weight, more preferred from 1.5% to 4% by weight of the composition, more preferably from 1.5% to 3.5% by weight. In such case (the composition in the form of a lotion) preferably the non-ionic surfactants comprise a non-ionic surfactant having a HLB value ranging from 2 to 6.5, preferably from 4 to 6, at a concentration ranging from 0.5% to 2%, preferably from 0.5% to 1.7% by weight, and/or a non-ionic surfactant having a HLB value ranging from 6.5 to 18, preferably from 12 to 18, at a concentration ranging from 0.5% to 2%, preferably from 0.5% to 1.2% by weight.

### Fatty Compound

A fatty compound has been defined herein before. The compositions prepared according to the method of the invention comprise a fatty compound having a melting point of at least 25° C. at a concentration of at least 1% by weight of the final composition. Preferably the concentration of fatty compounds ranges from 1% to 5% by weight of the final composition.

In one preferred embodiment the concentration of fatty compounds ranges from 1% to 4% by weight, preferably from 1% to 3.5% by weight, preferably from 1.5% to 3.5% by weight.

In another preferred embodiment the concentration of fatty compounds ranges from 2% to 5% by weight, preferably from 2% to 4.5% by weight, and preferably from 2% to 4% by weight.

In another preferred embodiment the concentration of fatty compounds ranges from 1% to 4% by weight, preferably from 1% to 3.5% by weight, preferably from 1.5% to 3.5% by weight, and the concentration of non-ionic surfactants ranges from 1% to 8% by weight, preferably from 1% to 4% by weight, preferably from 1% to 3.5% by weight, preferably from 1.5% to 3.5% by weight.

In another preferred embodiment the concentration of fatty compounds ranges from 2% to 5% by weight, preferably from 2% to 4.5% by weight, and preferably from 2% to 4% by weight, and the concentration of non-ionic surfactants ranges from 4% to 8% by weight, preferably from 4% to 7% by weight.

The fatty compound has a melting point of at least 25° C., preferably 30° C. or higher, preferably 40° C. or higher, more preferably 45° C. or higher, still more preferably 50° C. or higher, in view of stability of the composition obtained by the method of the invention.

Preferably, such melting point is up to about 90° C., more preferably up to about 80° C., still more preferably up to about 70° C., even more preferably up to about 65° C.

Preferably, fatty compounds having a melting point of at least 25° C. are selected from hydrocarbon oils, fatty esters or mixtures thereof. Straight chain hydrocarbon oils will preferably contain from about 12 to about 30 carbon atoms. Also suitable are polymeric hydrocarbons of alkenyl monomers, such as C₂-C₆ alkenyl monomers. Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols, monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are di- and triglyceride oils or fats, more specifically the di-, and tri-esters of glycerol and long chain carboxylic acids such as C₁₂-C₂₂ carboxylic acids. Preferred materials include cocoa butter, palm oil or fat, palm kernel oil or fat, palm oil fraction (e.g. palm stearin), and coconut oil or fat. Preferably the di- and triglyceride oils and fats are from vegetable origin.

More preferred, the fatty compound having a melting point of at least 25° C. is selected from one or more compounds from the group of fatty alcohols, triglyceride oils or fats, and mineral oils. Most preferred the fatty compound comprises a fatty alcohol, a fatty acid, a fatty alcohol derivative, or a fatty acid derivative, or mixtures thereof. A fatty alcohol derivative is a fatty alcohol which contains one or more side groups. A fatty acid derivative is a fatty acid which contains one or more side groups.

Fatty alcohols are typically compounds containing straight chain alkyl groups. The combined use of fatty alcohols and non-ionic surfactants in personal care compositions is believed to be especially advantageous, because this leads to the formation of a lamellar phase, in which the non-ionic surfactant is dispersed. The fatty alcohols useful herein are those preferably have from about 12 to about 30 carbon atoms. Preferably, the fatty alcohol comprises a C12-C22 fatty alcohol, preferably a C16-C22, more preferred a C16-C18 fatty alcohol. Preferably, these fatty alcohols are saturated and can be straight or branched chain alcohols.

Preferred fatty alcohols include, for example, cetyl alcohol (hexadecan-1-ol, having a melting point of about 56° C.), stearyl alcohol (1-octadecanol, having a melting point of about 58-59° C.), behenyl alcohol (having a melting point of about 71° C.), and mixtures thereof. In the present invention, more preferred fatty alcohols are cetyl alcohol, stearyl alcohol and mixtures thereof.

The level of fatty compound having a melting point of at least 25° C. in structured liquids prepared according to the method of the invention preferably ranges from 1 to 5%, preferably from 1% to 4.9% by weight. In case the composition is in the form of a cream (meaning a composition which is considered by users to be relatively thick), then preferably the concentration of fatty compounds ranges from 2% to 5% by weight of the composition, preferably from 2% to 4.9% by weight. Preferably the concentration ranges from 2% to 4.5% by weight, more preferred from 2% to 4% by weight of the composition.

In case the composition is in the form of a lotion (meaning a composition which is considered by users to be relatively thin), then preferably the concentration of fatty compounds having a melting point of at least 25° C. ranges from 1% to 4% by weight of the composition. Preferably the concentration ranges from 1% to 3.5% by weight, more preferred from 1.5% to 3.5% by weight of the composition.

Preferably, the weight ratio of the one or more non-ionic surfactants to the fatty compound having a melting point of at least 25° C. ranges from 5:1 to 0.5:1, preferably the ratio ranges from 4:1 to 0.6:1, preferably from 3:1 to 0.7:1.

### Other Ingredients

Preferably the total concentration of anionic surfactants, cationic surfactants, and zwitterionic surfactants is maximally 3% by weight in the compositions prepared according to the method of the invention. Preferably the total concentration of anionic surfactants, cationic surfactants, and zwitterionic surfactants is maximally 1% by weight, preferably maximally 0.5% by weight. Preferably the concentration of polymers is maximally 2% by weight, preferably maximally 1% by weight.

In addition to the fatty compound having a melting point of at least 25° C., also fatty compounds which are liquid at room temperature may be employed in the method of the invention to prepare a structured liquid. Preferred examples are liquid vegetable oils (such as sunflower oil, rapeseed oil, and soybean oil), and liquid mineral oils. These compounds may be present in order to provide an extra moisturising or smoothening effect on the skin when the structured liquid is used as a skin cream. Preferably the amounts of such liquid fatty compounds range from 0.5% by weight to 4% by weight, preferably from 0.5% by weight to 4% by weight

An antiperspirant active compound preferably is used in the method of the invention to prepare a structured liquid, preferably an aluminium compound and/or a zirconium compound. Such actives are water-soluble and are typically fully dissolved in the aqueous phase of the structured liquid. Preferably the antiperspirant active compound in a solution or dispersion in water is mixed in the method of the invention with the mixture from step a), preferably to a concentration in the composition ranging from 1 to 20% by weight, preferably from 3 to 15% by weight.

The antiperspirant active compound is typically selected from astringent salts, including both inorganic salts, salts with organic anions, and complexes. Preferred antiperspirant actives are aluminium, zirconium, and aluminium-zirconium chlorides, oxychlorides, and chlorohydrates salts. Particularly preferred antiperspirant actives are polynuclear in nature, meaning that the cations of the salt are associated into groups comprising more than one metal ion.

Aluminium halohydrates are especially preferred antiperspirant actives and may be defined by the general formula Al₂(OH)ₓQ_{y}.wH₂0, in which Q represents chlorine, bromine or iodine, x is variable from 2 to 5 and x+y=6 while wH₂O represents a variable amount of hydration. Aluminium chlorohydrate is the most preferred aluminium compound used as antiperspirant active. Aluminium chlorohydrate is a group of compounds having the general formula AlₙCl₍₃ₙ₋ₘ₎(OH)ₘ.

Zirconium salts are usually defined by the general formula ZrO(OH)₂₋ₓQₓ.wH2O in which Q represents chlorine, bromine or iodine; x is from about 1 to 2; w is from about 1 to 7; and x and w may both have non-integer values. Particular zirconium salts are zirconyl oxyhalides, zirconium hydroxyhalides, and combinations thereof.

Antiperspirant actives as used in the invention may be present as mixtures or complexes. Suitable aluminium-zirconium complexes often comprise a compound with a carboxylate group, for example an amino acid. Examples of suitable amino acids include tryptophan, beta-phenylalanine, valine, methionine, beta-alanine and, most preferably, glycine.

In some embodiments, it is desirable to employ complexes of a combination of aluminium halohydrates and zirconium chlorohydrates with amino acids such as glycine, which are disclosed in U.S. Pat. No. 3,792,068. Certain of these Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine.

Antiperspirant actives are preferably incorporated in an amount of from 0.5 to 60%, particularly from 5 to 30% or 40% and especially from 10% to 30% of the total composition.

Preferably the combination of non-ionic surfactant and fatty compound in the structured liquid forms a lamellar phase system in the composition. Such systems may be readily identified by means of optical microscopy or scanning electron microscopy. Such systems lead to good stability, particularly in compositions comprising an aluminium and/or zirconium containing antiperspirant active.

### Mixing in Controlled Deformation Dynamic Mixer

An advantage of the CDDM mixing device used in the method of the invention is that elongational and/or rotational shear flows can be controlled well, by modification of the rotational speed of one surface relative to the other. Moreover, also the distance between the two surfaces can be designed such that the flow field can be modified and adapted to the needs of the product to be produced by the CDDM. This leads to the advantage of the method of the invention, in that structured liquids are produced that have a relatively low concentration of active ingredients, especially the non-ionic surfactants and the fatty compound, while still being relatively high in viscosity. This results in saving on the amount of raw materials and resources required to make good and functional compositions.

The shear rate in the mixing apparatus is in the order of magnitude of at least 100,000 s⁻¹.

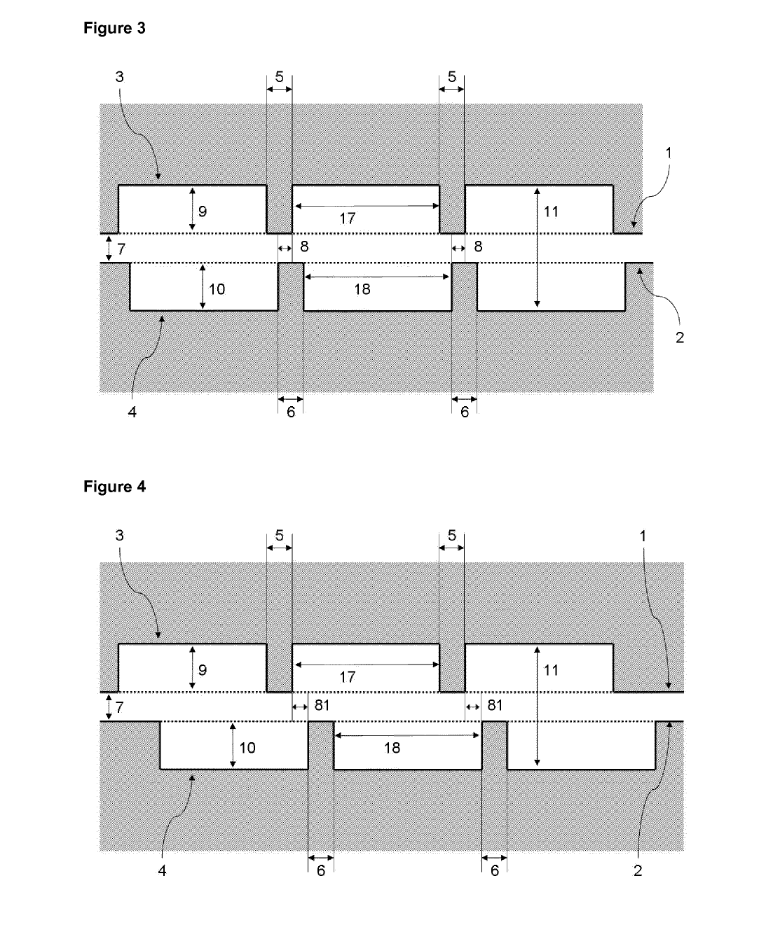

In a preferred embodiment the CDDM apparatus can be described by the following. With reference to FIG. 3 and FIG. 4, preferably the Controlled Deformation Dynamic Mixer comprises two confronting surfaces (1, 2), spaced by a distance (7),

wherein the first surface (1) contains at least three cavities (3), wherein at least one of the cavities has a depth (9) relative to the surface (1),
wherein the second surface (2) contains at least three cavities (4) wherein at least one of the cavities has a depth (10) relative to the surface (2),
wherein the cross-sectional area for flow of the liquid available during passage through the apparatus successively increases and decreases at least 3 times, and
wherein the surface (1) has a length (5) between two cavities, and
wherein the surface (2) has a length (6) between two cavities, and
wherein the surfaces (1, 2) are positioned such that the corresponding lengths (5, 6) overlap to create a slit having an offset distance (8) or do not overlap creating a offset distance (81),
wherein the cavities are arranged such that the cross-sectional area for flow of the liquid available during passage through the apparatus successively increases in the cavities and decreases in the slits by a factor of at least 5 and
wherein the distance (7) between the two surfaces (1,2) is between 2 micrometer and 300 micrometer, and wherein
either the ratio between the offset distance (8) and the distance (7) between the two surfaces (1, 2) ranges from 0 to 250,
or wherein the ratio between the offset distance (81) and the distance (7) between the two surfaces (1, 2) ranges from 0 to 30.

With reference to FIG. 3 and FIG. 4: there are several possible configurations for the mixing apparatus. In one preferred combination the confronting surfaces 1, 2 are cylindrical. In such a configuration the apparatus will generally comprise a cylindrical drum and co-axial sleeve. The confronting surfaces 1, 2 will be defined by the outer surface of the drum and the inner surface of the sleeve. However, there are alternative configurations in which the confronting surfaces are circular or disk-shaped. Between these two extremes of configuration are those in which the confronting surfaces are conical or frusto-conical. Non-cylindrical embodiments allow for further variation in the shear in different parts of the flow through the mixer.

The regions where the confronting surfaces 1, 2 are most closely spaced are those where the shear rate within the mixer tends to be the highest. The slit 7 between the surfaces between the confronting surfaces 1, 2 forms this region, combined with offset distance 8 or offset distance 81. High shear contributes to power consumption and heating. This is especially true where the confronting surfaces of the mixer are spaced by a gap of less than around 50 micrometer. Advantageously, confining the regions of high shear to relatively short regions means that the power consumption and the heating effect can be reduced, especially where in the CTM-like regions the confronting surfaces are spaced apart relatively widely.

Hence the apparatus can be designed such that good mixing is obtained, while keeping the pressure drop over the apparatus as small as possible. The design can be modified by adjusting the dimensions of the various parts of the apparatus, as explained in the following.

The distance 7 between the corresponding surfaces preferably is from 2 micrometers to 300 micrometers, which corresponds to the height of the slit. Preferably the distance 7 is between 3 micrometer and 200 micrometer, preferably between 5 micrometer and 150 micrometer, preferably between 5 micrometer and 100 micrometer, preferably between 5 micrometer and 80 micrometer, preferably between 5 and 60 micrometer, preferably between 5 micrometer and 40 micrometer. More preferably the distance 7 is between 8 micrometer and 40 micrometer, more preferably between 8 micrometer and 30 micrometer, more preferably between 10 micrometer and 30 micrometer, more preferably between 10 micrometer and 25 micrometer, more preferably between 15 micrometer and 25 micrometer. The actual height of the slit 7 depends on the dimensions of the apparatus and the required flow rate, and the skilled person will know how to design the apparatus such that the shear rates within the apparatus remain relatively constant irrespective of the size of the apparatus.

The surfaces 1 and 2 that each contain at least three cavities 3, 4 create a volume between the surfaces for flow of the two fluids which are mixed. The cavities in the surface effectively increase the surface area available for flow. Due to the presence of the cavities, the small area for flow between the surfaces 1 and 2 can be considered to be a slit having a height 7. The spacing 5 between two cavities in surface 1 and spacing 6 between two cavities in surface 2 and the relative position of these corresponding parts (the offset) determine the maximum length or offset distance 8 of the slit (in the direction of bulk liquid flow). The maximum length of the slit is equal to the smallest of the spacings 5, 6.

Preferably, the two surfaces 1, 2 with cavities 3, 4, that together form the volume for the mixing of the three phases (aqueous phase, liquid oil, and structuring fat), are positioned such that the corresponding spacings 5, 6 of the surfaces (that create the length of the slit) create an offset distance 8 of the slit (in the direction of the bulk flow) which is maximally 250 times as large as the distance 7 between the surfaces. The two surfaces 1, 2 can be positioned such that offset distance 8 can be adjusted. Preferably the ratio between the offset distance 8 and the distance 7 between the two surfaces 1, 2 ranges from 0 to 100, preferably 0 to 10, preferably 0 to 5. Most preferably the ratio between the offset distance 8 and the distance 7 ranges from 0 to 1. As an example, when the ratio between offset distance 8 and distance 7 is 5, and the distance 7 between the two surfaces 1, 2 is 15 micrometer, then the offset distance 8 of the slit is 75 micrometer.

Preferably and alternatively the surfaces 1, 2 are positioned such that no overlap is created, however in that case an offset distance 81 is created. In that case there is no overlap between the corresponding parts of the surfaces 1, 2, and the slit is created with what could be called a ‘negative overlap’. The two surfaces 1, 2 can be positioned such that offset distance 81 can be adjusted. The ratio between the offset distance 81 and the distance 7 between the two surfaces 1, 2 preferably ranges from 0 to 30. This ‘negative overlap’ accommodates the possibility of near zero distance 7 between the two corresponding surfaces 1 and 2. Preferably the offset distance 81 is such, that the ratio between the offset distance 81 and the distance 7 between the two surfaces 1, 2 ranges from 0 to 15, more preferred from 0 to 10, preferably from 0 to 5, preferably from 0 to 2 and more preferably from 0 to 1. Alternatively and preferably the offset distance 81 is maximally 600 micrometer, more preferably maximally 300 micrometer. As an example, when the ratio between length 81 and distance 7 is 2, and the distance 7 between the two surfaces 1, 2 is 15 micrometer, then length 81 (or what could be called negative overlap) is 30 micrometer.

A further benefit of this variation in the normal separation of the confronting surfaces in the direction of bulk flow, is that by having relatively small regions of high shear, especially with a low residence time is that the pressure drop along the mixer can be reduced without a compromise in mixing performance.

The little overlap (meaning that offset distance 8 approaches zero, or that the mixing apparatus has a ‘negative overlap’ or offset distance 81) between the corresponding parts of the surfaces 1, 2 leads to a relatively small pressure that is required in order to create a fine dispersion, as compared to apparatuses which have a longer overlap and consequently also need a higher pressure. Usually a longer distance of a slit (or longer capillary) leads to smaller droplets of the dispersed phase. Now we found that with a short capillary or even without capillary the droplets of the dispersed phase remains small, while the pressure required is relative low, as compared to a longer overlap. For example high pressure homogenisers may operate at pressures up to 1,600 bar or even higher. Hence preferably in the method of the invention, the mixing apparatus is operated at a pressure less than 200 bar, preferably less than 80 bar. In case the composition to be prepared has a relatively low viscosity, then the pressure is preferably less than 60 bar, preferably less than 40 bar, most preferred less than 30 bar. With these relatively low pressures a good mixing process is obtained.

An additional advantage of the relatively low pressure is that the energy consumption for applying the pressure is much lower than in devices like high pressure homogeniser which may use pressures of up to 1,000 bar. Moreover less stringent material specifications for design of an apparatus to withstand high pressures is required, such that raw materials can be saved.

With reference to FIG. 3 and FIG. 4, the fluids preferably flow from left to right through the apparatus. The slits create an acceleration of the flow, while at the exit of the slit the fluids decelerate due to the increase of the surface area for flow and the expansion which occurs. The acceleration and deceleration leads to the break up of the large droplets of the dispersed phase, to create finely dispersed droplets in a continuous phase. Droplets that are already small, remain relatively untouched. The flow in the cavities is such that the droplets of the dispersed phase eventually become evenly distributed in the continuous phase.

The cross-sectional area for flow of the liquid available during passage through the apparatus successively increases and decreases at least 5 times, and these passages lead to effective mixing of the two fluids. This means that the cross-sectional area for flow of liquid in the cavities is at least 5 times larger than the cross-sectional area for flow of liquid in the slits. This relates to the ratio between distance 11 and distance 7. Preferably the cross-sectional area for flow is designed such that the cross-sectional area for flow of the liquid available during passage through the apparatus successively increases and decreases by a factor of at least 7, preferably at least 10, preferably at least 25, preferably at least 50, up to preferred values of 100 to 400. The cross-sectional surface area for flow of the fluids is determined by the depth 9 of the cavities 3 in the first surface 1 and by the depth 10 of the cavities 4 in the second surface 2. The total cross-sectional area is determined by the distance 11 between the bottoms of two corresponding cavities in the opposite surfaces.

The surfaces 1, 2 each contain at least three cavities 3, 4. In that case the flow expands at least 3 times during passage, and the flow passes through at least 3 slits during the passage. Preferably the cross-sectional area for flow of the liquid available during passage through the apparatus successively increases and decreases between 4 and 8 times. This means that the flow during passage experiences the presence of between 4 and 8 slits and cavities.

The shape of the cavities 3 may take any suitable form, for example the cross-section may not be rectangular, but may take the shape of for example a trapezoid, or a parallelogram, or a rectangle where the corners are rounded. Seen from above, the cavities may be rectangular, square, or circular, or any other suitable shape. Any arrangement of the cavities and the number of cavities and size of the cavities may be within the scope of the present invention.

The mixing apparatus preferably is operated dynamically, meaning that the confronting surfaces 1, 2 are relatively moveable. In case the apparatus is designed such that the confronting surfaces 1, 2 are cylindrical, and the apparatus comprises a cylindrical drum and co-axial sleeve, then preferably the cylindrical drum is able to rotate. In that case preferably one of the surfaces rotates relative to the other surface at a frequency between 1,000 and 25,000 rotations per minute, preferably between 3,000 and 12,000 rotations per minute. Preferably the cylindrical drum rotates at a frequency between 1,000 and 25,000 rotations per minute, preferably between 3,000 and 12,000 rotations per minute.

As indicated before, in another preferred embodiment the surfaces are static with respect to each other. That means that during the mixing operations the liquid is pressed through the mixing apparatus, and the surfaces or cylindrical drum do not rotate.

In general rotation may lead to improved mixing process. Static operation has the advantage that less energy is required for mixing. Operation of the device without rotation leads to very efficient and effective mixing of fluids. The static operation enjoys the major advantage of potentially easier deployment and less mechanical complexity and possibly wear of equipment. The dynamic operation has the advantage that the required pressure to pump one or more fluids through the device, is lower than at the static operation.

Additional features of the known CTM and CDDM may be incorporated in the mixer described herein. For example, one or both of the confronting surfaces may be provided with means to heat or cool it. Where cavities are provided in the confronting surfaces these may have a different geometry in different parts of the mixer to as to further vary the shear conditions.

In a preferred example, the dimensions of such a CDDM apparatus used in the invention are such that the distance between the two surfaces 7 is between 10 and 20 micrometer; and/or wherein the length of the slit 8 is maximally 2 millimeter, for example 80 micrometer, or 20 micrometer, or even 0 micrometer. The length of the slit 8 plus the length of the cavity 17, 18 combined is maximally 10 millimeter; and/or wherein the depth of the cavities 9, 10 is maximally 2 millimeter. In that case preferably the internal diameter of the outer surface is between 20 and 30 millimeter, preferably about 25 millimeter. The total length of the apparatus in that case is between 7 and 13 centimeter, preferably about 10 centimeter. The length means that this is the zone where the fluids are mixed. The rotational speed of such a preferred apparatus is preferably 0 (static), or more preferred alternatively between 5,000 and 25,000 rotations per minute.

The shape of the area for liquid flow may take different forms, and naturally depends on the shape of the confronting surfaces. If the surfaces are flat, then the cross-sectional area for flow may be rectangular. The two confronting surfaces may also be in a circular shape, for example a cylindrical rotor which is positioned in the centre of a cylindrical pipe, wherein the outside of the cylindrical rotor forms a surface, and the inner surface of the cylindrical pipe forms the other surface. The circular annulus between the two confronting surface is available for liquid flow.

Structured Liquids and Use of these as Personal Care Composition

In a second aspect the present invention provides a structured liquid obtainable by the method according to the invention. The second aspect of the present invention also provides a structured liquid obtained by the method according to the invention. The structured liquid composition that is obtainable by the method of the invention, or obtained by the method of the invention preferably have a composition as indicated in the following paragraphs. The preferred non-ionic surfactants and fatty compounds as indicated in the context of the first aspect of the invention are also applicable in this second aspect of the invention, mutatis mutandis.

These structured liquids have the advantage that they have a relatively low concentration of active ingredients, especially the non-ionic surfactants and the fatty compound, while still being relatively high in viscosity. This results in saving on the amount of raw materials and resources required to make good and functional compositions.

### Lotion Structured Liquid Composition

The second aspect of the invention also provides a structured liquid composition comprising water, and one or more fatty compounds at a concentration ranging from 1% to 4% by weight, and

one or more non-ionic surfactants at a concentration ranging from 1% to 8% by weight, and water,
and wherein the total concentration of anionic surfactants, cationic surfactants, and zwitterionic surfactants is maximally 3% by weight,
and wherein the structured liquid has a dynamic viscosity of at least 80,000 mPa·s, preferably at least 100,000 mPa·s, measured using a Brookfield RV viscometer, fitted with a T-bar T-E spindle, at a rotational speed of 5 rpm, and a temperature of 25° C. The dynamic viscosity value is determined by performing the actual measurement 1 minute after initiating the measurement procedure, as the composition needs to equilibrate in the viscometer. This composition, with the concentration of actives and the viscosity as specified, is considered by the user of this composition to be a lotion for personal care, for example for use as a skin lotion or a deodorant lotion or an antiperspirant lotion.

Preferably the concentration of fatty compounds in this structured liquid composition ranges from 1% to 3.5% by weight, preferably from 1.5% to 3.5% by weight. Preferably the concentration of non-ionic surfactants ranges from 1% to 6% by weight, preferably from 1.5% to 4% by weight., preferably from 1.5% to 3.5% by weight.

Preferably this structured liquid composition is a composition wherein the one or more non-ionic surfactants comprise a non-ionic surfactant having a HLB value ranging from 2 to 6.5, preferably from 4 to 6, at a concentration ranging from 0.5% to 7%, preferably from 0.5% to 5% by weight, preferably from 0.5% to 3%, and most preferred from 0.5% to 1.7% by weight, and/or a non-ionic surfactant having a HLB value ranging from 6.5 to 18, preferably from 12 to 18, at a concentration ranging from 0.5% to 2%, preferably from 0.5% to 1.2% by weight.

Preferably this structured liquid composition comprises at least 72% by weight water, preferably at least 80% by weight water, preferably at least 85% by weight, preferably at least 88% by weight, preferably at least 90% by weight, and most preferably at least 92% water by weight of the composition.

In case the structured liquid composition comprises an anti-perspirant active compound, then the composition comprises preferably at least 60% water, preferably at least 67% water, preferably at least 75% water, more preferred at least 80% water by weight of the composition.

The dynamic viscosity of this composition is at least 80,000 mPa·s (80 Pa·s), preferably at least 100,000 mPa·s, preferably at least 130,000 mPa·s, preferably at least 150,000 mPa·s. Even more preferred the dynamic viscosity of the composition is at least 200,000 mPa·s. Preferably these dynamic viscosities are measured using a Brookfield RV viscometer, fitted with a T-bar T-E spindle, at a rotational speed of 5 rpm and a temperature of 25° C. The dynamic viscosity value is determined by performing the actual measurement 1 minute after initiating the measurement procedure, as the composition needs to equilibrate in the viscometer.

### Cream Structured Liquid Composition

The second aspect of the invention also provides a structured liquid composition comprising water, and one or more fatty compounds at a concentration ranging from 2% to 5% by weight, and

one or more non-ionic surfactants at a concentration ranging from 4% to 8% by weight, and water,
and wherein the total concentration of anionic surfactants, cationic surfactants, and zwitterionic surfactants is maximally 3% by weight,
and wherein the structured liquid has a dynamic viscosity of at least 60,000 mPa·s, preferably at least 80,000 mPa·s, measured using a Brookfield RV viscometer, fitted with a T-Bar T-D spindle at a rotational speed of 10 rpm, and a temperature of 25° C. The dynamic viscosity value is determined by performing the actual measurement 1 minute after initiating the measurement procedure, as the composition needs to equilibrate in the viscometer. This composition, with the concentration of actives and the viscosity as specified, is considered by the user of this composition to be a cream for personal care, for example for use as a skin cream or a deodorant cream or an antiperspirant cream.

Preferably the concentration of fatty compounds in this structured liquid composition ranges from 2% to 4.9% by weight, preferably from 2% to 4.5% by weight, preferably from 2% to 4% by weight. Preferably the concentration of non-ionic surfactants ranges from 4% to 7% by weight. Preferably this structured liquid composition is a composition wherein the one or more non-ionic surfactants comprises a non-ionic surfactant having a HLB value ranging from 2 to 6.5, preferably from 4 to 6, at a concentration ranging from 3% to 7%, preferably from 3% to 6% by weight, and/or

a non-ionic surfactant having a HLB value ranging from 6.5 to 18, preferably from 12 to 18, at a concentration ranging from 0.5% to 3%, preferably from 1% to 2.5% by weight.

Preferably this structured liquid composition comprises at least 72% by weight water, preferably at least 80% by weight water, preferably at least 83% by weight, preferably at least 85% by weight, preferably at least 90% by weight, preferably at least 92% water by weight of the composition.

In case the structured liquid composition comprises an anti-perspirant active compound, then the composition comprises preferably at least 64% water, preferably at least 71% water, preferably at least 75% water, more preferred at least 78% water by weight of the composition.

The dynamic viscosity of this composition is at least 60,000 mPa·s (60 Pa·s), preferably at least 80,000 mPa·s, preferably at least 100,000 mPa·s, preferably at least 120,000 mPa·s. Preferably these dynamic viscosities are measured using a Brookfield RV viscometer, fitted with a T-bar T-D spindle, at a rotational speed of 10 rpm and a temperature of 25° C. The dynamic viscosity value is determined by performing the actual measurement 1 minute after initiating the measurement procedure, as the composition needs to equilibrate in the viscometer.

Preferably the structured liquid compositions according to the second aspect of the invention are compositions wherein the concentration of anionic surfactants is maximally 3% by weight. Preferably the concentration of cationic surfactants is maximally 3% by weight. Preferably the concentration of zwitterionic surfactants is maximally 3% by weight. Preferably, the total concentration of anionic surfactants, cationic surfactants, and zwitterionic surfactants is maximally 1% by weight, preferably maximally 0.5% by weight. Preferably the concentration of anionic surfactants is maximally 1% by weight, preferably less than 1% by weight, preferably maximally 0.5% by weight, preferably less than 0.5% by weight. Preferably any or all of the anionic surfactants, cationic surfactants, and zwitterionic surfactants are absent from the composition.

The compositions according to the second aspect of the invention preferably comprise polymers at a maximum concentration of 2% by weight, preferably maximally 1% by weight. Preferably the maximum concentration of polymers is 0.5% by weight, preferably maximally 0.2% by weight, or even maximally 0.1% by weight. Most preferred polymers are absent from the compositions of the invention. Polymers in the context of the invention may be any polymer commonly used in personal care compositions. They may comprise proteins like gelatin or milk proteins like casein, caseinate, and whey protein. They may comprise polysaccharides like hydrocolloid thickeners, for example gums like guar gum, xanthan gum, locust bean gum, and gum arabic, or for example cellulosic materials. They may also comprise polyethylene glycols, preferably containing 30 ethylene glycol moieties or more. The may also comprise synthetic polymers like polyethylene, or polyacrylates, polymethacrylates, or copolymers containing monomers like the acrylates or methacrylates. The polymers may be neutral, or may be charged like anionic polymers, or cationic polymers, or zwitterionic polymers. The polymers may also comprise blends of these exemplified polymers.

The compositions of the invention preferably comprise silicone compounds, at a concentration ranging from 0.1% to 2% by weight. These compounds may provide benefit to the skin. The compositions of the invention preferably comprise glycerol as smoothener and for lubrication and as humectant, at a concentration ranging from 0.5% to 10% by weight, preferably from 0.5% to 5% by weight, preferably from 0.5% to 4% by weight.

Preferably the concentration of water-soluble or water-dispersible thickening agents like proteins, mono-, di-, oligo- and polysaccharides; cellulosic materials, gums, clays, or blends or derivatives thereof is maximally 2% by weight, preferably maximally 1% by weight. Preferably the maximum concentration of these compound is 0.5% by weight, preferably maximally 0.2% by weight, or even maximally 0.1% by weight. Most preferred these compounds are absent from the compositions of the invention.

Preferably the structured liquid composition according to the second aspect of the invention comprises an antiperspirant active, preferably comprising an aluminium compound and/or a zirconium compound. In that case the composition can be used as a deodorant or an antiperspirant. The second aspect of the invention also provides a product for treating perspiration comprising a composition prepared according to the method of the first aspect of the invention and comprising an antiperspirant active, preferably comprising an aluminium compound and/or a zirconium compound, or according to second aspect of the invention, and an applicator comprising a reservoir for holding the composition and a surface for applying the composition to the skin. A preferred applicator comprises a reservoir for holding the composition, and a base that can be twisted to extrude the composition to the top of the applicator. By the extrusion the composition is moved to the top surface, and with this surface the composition can be applied to the skin. The compositions may also be sold in packages like sachets or bottles, and can be used as a skin lotion or a skin cream.

In a third aspect the present invention provides use of a structured liquid, prepared according to the method of first aspect of the invention and comprising an antiperspirant active, preferably comprising an aluminium compound and/or a zirconium compound, or according to the second aspect of the invention as deodorant or antiperspirant.

### EXAMPLES

The following non-limiting examples illustrate the present invention.

### CDDM Apparatus

Experiments were carried out in a CDDM apparatus as schematically depicted in FIG. 2 and FIG. 3, wherein the apparatus comprises a stainless steel cylindrical drum and co-axial sleeve (the confronting surfaces 1, 2 are cylindrical). The confronting surfaces 1, 2 are defined by the outer surface of the drum and the inner surface of the sleeve, respectively. The CDDM can be described by the following parameters:
slit height 7 is 35-40 micrometer;
offset distance 8 is 20 micrometer;
total length of the apparatus is 10 centimeter (length means the zone where the fluids are mixed);
across the length of the CDDM in axial direction (in flow direction) the flow experiences six slits with height 7, the flow is contracted 6 times;
depth 9, 10 of cavities 3, 4 is maximally 2 millimeter;
internal diameter of the stator is 25 millimeter;
rotational speed of the apparatus is up to 25,000 rotations per minute, and it was operated in these experiments at 2,000 to 18,000 rotations per minute;

### Raw Materials

TABLE 1
Raw material description as used in the examples.

| Chemical (INCI) | Trade name and | |
|---|---|---|
| name | supplier | Functionality |
| Cetearyl | Polawax GP200, | fatty compound (mixture of fatty |
| alcohol, | ex Croda | alcohols, predominantly cetyl |
| PEG-20 | | and stearyl alcohols), and non- |
| stearate | | ionic surfactant with HLB ~15 |
| Glyceryl | Cutina GMS-V, | Glyceryl mono stearate mono- |
| stearate | Cutina MD, | glyceride, non-ionic surfactant, |
| | ex Cognis | HLB ~3.8, melting point |
| | | 48-56° C. |
| Cetearyl alcohol, | Lanette O, | fatty compound (mixture of |
| (ceto-stearyl | ex BASF | fatty alcohols, predominantly |
| alcohol) | | cetyl and stearyl alcohols) |
| Stearyl alcohol | Lanette C18, | fatty compound (stearyl alcohol) |
| | ex BASF | |
| White | Blandol, ex | fatty compound (mineral oil) |
| mineral oil | Sonneborn | |
| Phenoxyethanol | Phenoxetol, | preservative |
| | ex Clariant | |
| Iodopropynyl | Glycacil L, | preservative |
| Butylcarbamate | ex Lonza | |
| Steareth-20 | Brij S20, | non-ionic surfactant, polyethylene |
| | Brij 78, | glycol (n = 20) octadecyl |
| | ex Croda | ether, HLB ~15.3, melting |
| | | point 44-46° C. |
| Aluminium | Chlorohydrol | deodorant/antiperspirant active |
| Chlorohydrate | 50% solution | |
| | ACH-50, ex | |
| | Reheis | |
| Glycerol | Palmera G995V, | For smoothness, lubrication, |
| | ex KLK Oleo | humectant |
| Fragrance | | Fragrance |

The Polawax GP200 was analysed on its content of cetearyl alcohol, and this amount was about 80% by weight. The method was a combination of gas chromatography with mass spectrometry (GC-MS). It is assumed here that the amount of PEG-20 stearate is 20% by weight.

### Characterisation of Viscosities

The viscosities for structured liquids were determined using a Brookfield RV viscometer (ex Brookfield Engineering Laboratories, Inc., Middleboro, Mass., USA), fitted with a T-Bar T-D or T-E spindle and operated at a rotational speed of 0.5 rpm to 10 rpm, and at room temperature between about 15 and 25° C. Whenever viscosity is mentioned in here, the dynamic viscosity (in mPa·s or Pa·s) is meant.

### Determination of Yield Stress

The yield stress is defined as the minimum stress for creep to take place (The Penguin Dictionary of Physics, Penguin; 3^{rd} revised edition, 2004). Below this value any deformation produced by an external force will be purely elastic. It is directly determined to characterize the overall strength of a composition before flow. For products where there is a yield stress, identified from the shape of the flow curve (obtained from a plot of viscosity versus shear stress), for convenience, the yield stress is taken as being the shear stress evaluated at the point where the shear rate=0.1 s⁻¹.

Used rheometer: Anton Paar DSR 300 with profiled cup (CC27) and vane and basket geometry (ST14-4V-3S), which is equivalent to concentric cylinders. Measurement type: stepped stress (for flow curve under controlled stress mode). Protocol:
600 s stand by;
logarithmic ramp: from applied shear stress of 5 Pa to 700 Pa (with an event-controlled stop if the system overspeeds, for Anton Paar rheometers the rotational speed limit is 1190 rpm);
60 points per decade;
temperature: 25° C.

The output is the apparent viscosity (in Pa·s) as function of applied shear stress (in Pa). This is a curve which generally starts at a high value and at a certain shear stress the apparent viscosity decreases rapidly. The yield stress is the value of shear stress where the apparent viscosity drops at its highest rate.

### Example 1

### Preparation of Structured Liquid Compositions Using CDDM

A structured liquid personal care composition was prepared as per the formulation and process instructions detailed below.

TABLE 2
Composition of structured liquid composition.

| | | concentration |
|---|---|---|
| Ingredient (INCI name) | Trade name | [wt %] |
| Aqua | Demineralised water | 86.03 |
| Cetearyl alcohol and PEG-20 | Polawax GP200 | 5.00 |
| stearate | | |
| Glyceryl stearate | Cutina GMS-V | 7.50 |
| Cetearyl alcohol | Lanette O | 1.00 |
| Phenoxyethanol | Phenoxetol | 0.40 |
| Iodopropynyl butylcarbamate | Glycacil L | 0.07 |

The process that was used to prepare these concentrated liquid compositions was the following. The mixing equipment that was used was a Fryma DT10, which is a mixed vessel with jacketed chamber to control temperature in the vessel. The vessel was equipped with a Cowles dispersion disc impeller.
1. Fatty compounds and non-ionics were added to a side vessel and heated to 80° C.
2. Demineralised water was added into main vessel and heated to 80° C.
3. The compounds from step 1 were added to the main vessel once they had melted and were at 80° C., while the contents were mixed with the impeller at 400 rpm.
4. The batch was cooled to 50° C. under shear while mixing with impeller at 400 rpm. Batch thickened quickly.
5. Preservatives were added to main vessel.
6. Contents were cooled to 40° C. and mixed for 15 minutes before discharging.

This composition was the control sample prepared according to a standard method, having what is called the 100% concentration of actives. This mixture was used to be fed into the CDDM apparatus as already described above. The CDDM was operated statically, meaning that the rotor did not rotate. The flow rate of the mixture was set at 20 or 80 milliliter per second (=72 or 288 liter per hour). After the composition had been passed through the CDDM, it was diluted with water to a composition having a concentration of active compounds of 87.5% of the starting point (based on weight). This dilution was done by mixing water with the composition, using a vessel equipped with a paddle stirrer, rotating gently. This diluted composition was passed through the CDDM. Similarly this composition was again diluted to a concentration of active compounds of 75% of the starting point (based on weight), and a further dilution to a concentration of active compounds of 62.5% of the standard (based on weight).

These dilutions indicate the concentrations of active components in the various compositions, wherein 100% is the reference in Table 2. This gives the following concentration of fatty compounds and non-ionic surfactants in the compositions.

TABLE 3
Concentration of fatty compounds and non-ionic
surfactants, based on composition from Table 2,
for non-diluted and two diluted compositions.

| | 100% | 75% diluted | 62.5% diluted |
|---|---|---|---|
| | composition | composition | composition |
| Fatty compounds: | 1 + 4 = 5 | 3.75 | 3.13 |
| Low HLB (2-6.5) | 7.5 | 5.63 | 4.69 |
| Non-ionic surfactants | | | |
| High HLB (6.5-18) | 1 | 0.75 | 0.63 |
| Non-ionic surfactants | | | |
| Total Non-ionic | 8.5 | 6.38 | 5.31 |
| surfactants: | | | |

The same procedure was followed with a CDDM, with the rotating member rotating at a speed of 10,000 rpm. Similarly the various compositions (non-diluted and diluted) were passed through the CDDM device at flow rates of 20 or 80 milliliter per second.

The compositions that had been passed through the CDDM were compared to control samples at the same dilutions that were not passed through the CDDM.

Each of these various dilutions of the structured liquid coming out of the CDDM and the control samples were subjected to rheology measurements. A Brookfield RV viscometer (ex Brookfield Engineering Laboratories, Inc., Middleboro, Mass., USA) fitted on a Helipath stand was used to determine the dynamic viscosity of the various samples. The viscometer was fitted with a T-Bar T-E spindle and operated at a rotational speed of 10 rpm, and at a temperature of 25° C. The dynamic viscosity value is determined by performing the actual measurement 1 minute after initiating the measurement procedure, as the composition needs to equilibrate in the viscometer.

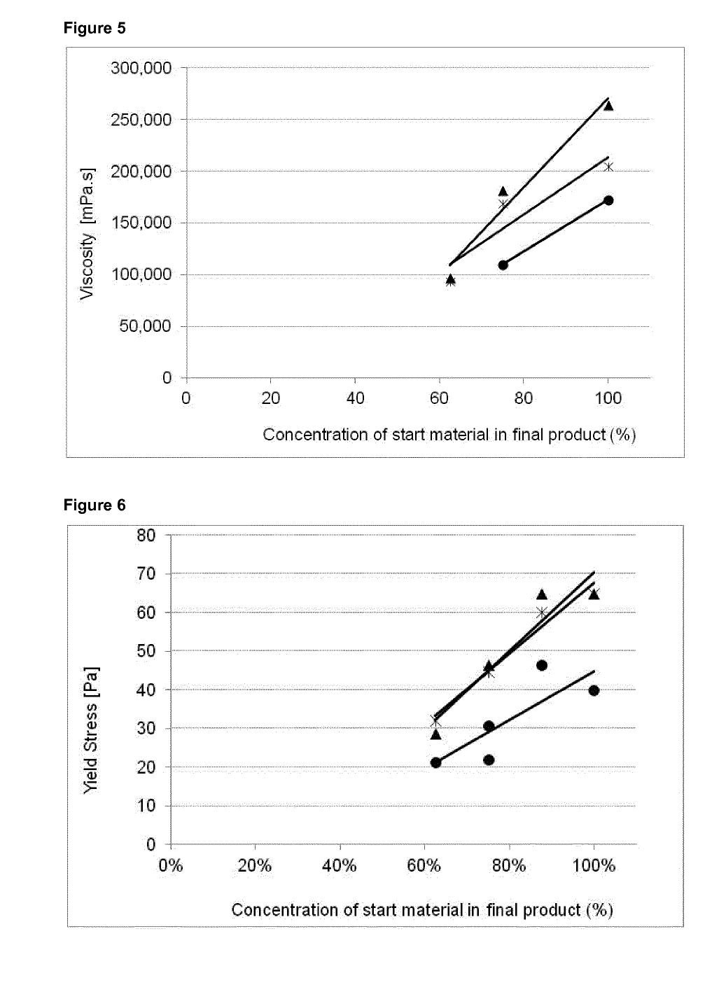

FIG. 5 plots the control samples (, not passed through the CDDM), and the samples that have been passed through the CDDM, operated in either rotating mode (▴) or static mode (*). It is shown here that the dynamic viscosity strongly increases when the samples have passed the CDDM. A concentration of actives of about 75% seems to give a similar dynamic viscosity as the control sample at 100% concentration. Whether the CDDM is operated rotating or static seems to have some influence, mainly at the 100% value.

The yield stress of the various samples as function of the degree of dilution is plotted in FIG. 6. This figure plots the control samples (, not passed through the CDDM), and the samples that have been passed through the CDDM, operated in either rotating mode (▴) or static mode (*). The yield stress of the samples that have been passed through the CDDM is higher than of the control samples. A concentration of actives of about 75% seems to give a similar yield stress as the control sample at 100% concentration. Moreover, the rotation of the CDDM does not seem to influence the yield stress. For all samples holds that the yield stress seems to be linearly related to the concentration of the actives in the composition. The yield stress of the samples that passed the CDDM increases with a higher rate as function of the concentration of actives, than the control sample.

This example shows that the viscosity and yield stress of the product which is mixed using CDDM is much higher than the product prepared in a conventional way, with the same concentration of actives. Or in other words, with a reduced concentration of actives, the same dynamic viscosity, viscosity profile as function of shear stress, and yield stress can be obtained as a product produced without mixing in the CDDM. In this case the concentration of actives can be decreased by about 25-30%, while keeping the same rheological properties.

### Example 2

### Preparation of Structured Liquid Deo Cream Compositions Using CDDM

A structured liquid composition was prepared having the composition as in the following table, and the preparation method similarly as in example 1. In this experiment the composition contained the antiperspirant active aluminium chlorohydrate, as well as glycerol.

TABLE 4
Composition of structured liquid composition - deo cream

| | | Concentration |
|---|---|---|
| Ingredient (INCI name) | Trade name | [wt %] |
| Aqua | Demineralised water | 52.33 |
| Aluminium chlorohydrate | ACH-50 | 30.00 |
| Glycerol | Palmera G995V | 1.50 |
| Cetearyl alcohol and | Polawax GP200 | 5.00 |
| PEG-20 stearate | | |
| Glyceryl stearate | Cutina GMS-V | 7.50 |
| Cetearyl alcohol | Lanette O | 1.00 |
| White mineral oil | Blandol | 1.00 |
| Fragrance | | 1.20 |
| Phenoxyethanol | Phenoxetol | 0.40 |
| Iodopropynyl butylcarbamate | Glycacil L | 0.07 |

This composition was produced similarly as described in example 1. The composition was split in various parts, and each part was diluted with water to different concentrations. Diluting was done by mixing the control sample with water at ambient temperature using a paddle stirrer which was rotating gently. The dilutions that were prepared were: 87.5%, 75%, 62.5%, and 50% of the amount of actives as the control sample. This gives the following concentration of fatty compounds and non-ionic surfactants in the compositions, as based on Table 4.

TABLE 5
Concentration of fatty compounds and non-ionic
surfactants, based on composition from Table 4,
for non-diluted and two diluted compositions.

| | 100% | 75% diluted | 62.5% diluted |
|---|---|---|---|
| | composition | composition | composition |
| Fatty compounds: | 1 + 1 + 4 = 6 | 4.50 | 3.75 |
| Low HLB (2-6.5) | 7.5 | 5.63 | 4.69 |
| Non-ionic surfactants | | | |
| High HLB (6.5-18) | 1 | 0.75 | 0.63 |
| Non-ionic surfactants | | | |
| Total Non-ionic | 8.5 | 6.38 | 5.31 |
| surfactants: | | | |

These compositions were fed into the CDDM apparatus as described before. The CDDM was operated in static mode, meaning that the rotor did not rotate. The flow rate of the premix was set at 80 milliliter per second (=288 liter per hour). This same procedure was followed with a CDDM, with the rotating member rotating at a speed of 10,000 rpm. Similarly the various compositions (non-diluted and diluted) were passed through the CDDM device at a flow rate of 80 milliliter per second.

The compositions that had been passed through the CDDM were compared to control samples at the same dilutions that were not passed through the CDDM. The basis for the control sample is the composition as described in Table 4 and prepared in the batch vessel described above.

Each of these various dilutions of the structured liquid coming out of the CDDM were subjected to rheology measurements. A Brookfield RV viscometer (ex Brookfield Engineering Laboratories, Inc., Middleboro, Mass., USA) fitted on a Helipath stand, was used to determine the dynamic viscosity of the various samples. The viscometer was fitted with a T-Bar T-D spindle and operated at a rotational speed of 10 rpm, and at a temperature of 25° C. The dynamic viscosity value is determined by performing the actual measurement 1 minute after initiating the measurement procedure, as the composition needs to equilibrate in the viscometer.

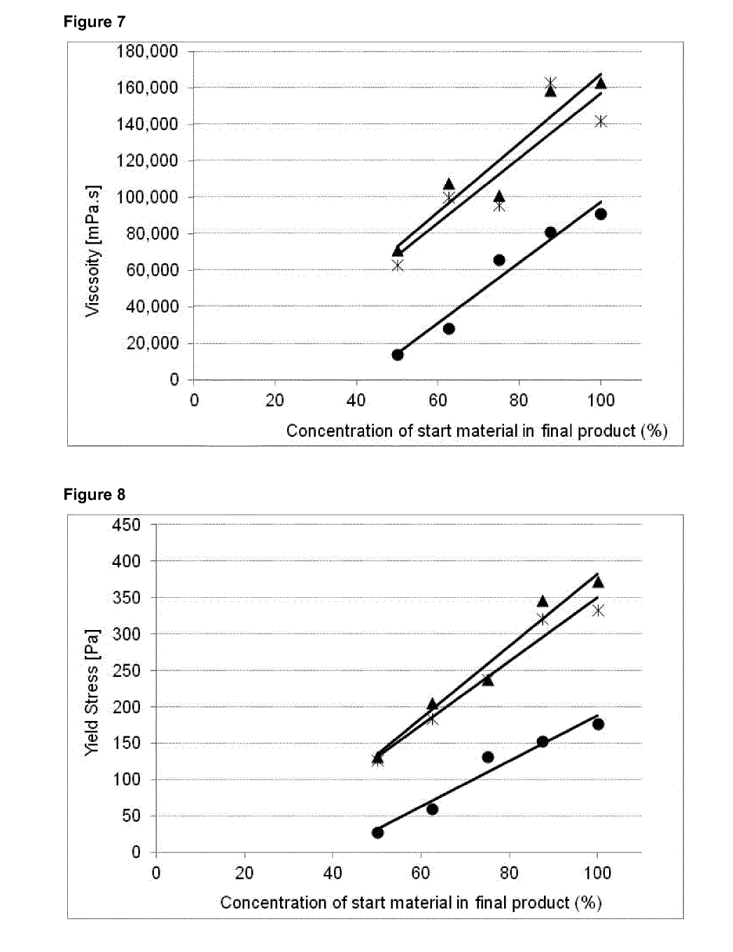

FIG. 7 plots the control samples (, not passed through the CDDM), and the samples that have been passed through the CDDM, operated in either rotating mode (▴) or static mode (*). It is shown here that the dynamic viscosity strongly increases when the samples have passed the CDDM. A concentration of actives of about 62-75% seems to give a similar dynamic viscosity as the control sample at 100% concentration. Whether the CDDM is operated rotating or static does not seem to make a big difference. The viscosity linearly increases with the concentration of actives.

The yield stress of the various samples as function of the degree of dilution is plotted in FIG. 8. This figure plots the control samples (, not passed through the CDDM), and the samples that have been passed through the CDDM, operated in either rotating mode (▴) or static mode (*). The yield stress of the samples that have been passed through the CDDM is higher than of the control samples. A concentration of actives of about 62% seems to give a similar yield stress as the control sample at 100% concentration. Moreover, the rotation of the CDDM does not seem to influence the yield stress. For all samples holds that the yield stress seems to be linearly related to the concentration of the actives in the composition. The yield stress of the samples that passed the CDDM, increases with a higher rate as function of the concentration of actives, than the control sample.

The presence of the aluminium compounds in the formulation in this example may influence the yield stress and the dynamic viscosity, due to ionic interactions of the aluminium chlorohydrate with other compounds in the formulation.

This example shows that the viscosity of the product which is mixed using CDDM is much higher than the product prepared in a conventional way, with the same concentration of actives. Or in other words, with a reduced concentration of actives, the same dynamic viscosity, viscosity profile as function of shear stress, and yield stress can be obtained as a product produced without mixing in the CDDM. In this case the concentration of actives can be decreased by about 25-38%, while keeping the same rheological properties.

### Example 3

### Deo Lotion Formulation

Structured liquids were produced containing the antiperspirant active aluminium chlorohydrate. This specific structured liquid was considered to be a deo lotion formulation.

The composition is specified in the following table.

TABLE 6
Formulations of structured liquid compositions - deo lotion.

| | | concentration |
|---|---|---|
| Ingredient (INCI name) | Trade name | [wt %] |
| Aqua | Demineralised water | 61.19 |
| Aluminium chlorohydrate | Chlorohydrol 50% solution | 30.00 |
| Glyceryl stearate | Cutina GMS-V | 2.00 |
| Stearyl alcohol | Lanette O18 | 3.50 |
| Cetearyl alcohol | Lanette O | 1.00 |
| Steareth - 20 | Brij 78 | 1.31 |
| Fragrance | | 1.00 |

This composition was produced similarly as described in example 1. The dilutions from this control sample were made similarly as in example 2, namely by splitting the composition in several parts and diluting each part. Dilutions with 87.5%, 75%, 62.5%, and 50% of the amount of actives as the control sample were prepared. This gives the following concentration of fatty compounds and non-ionic surfactants in the compositions, as based on Table 6.

TABLE 7
Concentration of fatty compounds and non-ionic
surfactants, based on composition from Table 6,
for non-diluted and two diluted compositions.

| | 100% | 75% diluted | 62.5% diluted |
|---|---|---|---|
| | composition | composition | composition |
| Fatty compounds: | 1 + 3.5 = 4.5 | 3.38 | 2.81 |
| Low HLB (2-6.5) | 2 | 1.50 | 1.25 |
| Non-ionic surfactants | | | |
| High HLB (6.5-18) | 1.31 | 0.98 | 0.82 |
| Non-ionic surfactants | | | |
| Total Non-ionic | 3.31 | 2.48 | 2.07 |
| surfactants: | | | |

These compositions were fed into the CDDM apparatus as described before. The CDDM was operated in static mode, meaning that the rotor did not rotate. The flow rate of the premix was set at 80 milliliter per second (=288 liter per hour). This same procedure was followed with a CDDM, with the rotating member rotating at a speed of 10,000 rpm. Similarly the various compositions (non-diluted and diluted) were passed through the CDDM device at a flow rate of 80 milliliter per second.

The compositions that had been passed through the CDDM were compared to control samples at the same dilutions that were not passed through the CDDM. The basis for the control sample is the composition as described in Table 6 and prepared in the batch vessel described above.

Each of these various dilutions of the structured liquid coming out of the CDDM were subjected to rheology measurements. A Brookfield RV viscometer (ex Brookfield Engineering Laboratories, Inc., Middleboro, Mass., USA), was used to measure the dynamic viscosity of the various samples. The viscometer was fitted with a T-Bar T-E spindle and operated at a rotational speed of 5 rpm, and at a temperature of 25° C. The dynamic viscosity value is determined by performing the actual measurement 1 minute after initiating the measurement procedure, as the composition needs to equilibrate in the viscometer.

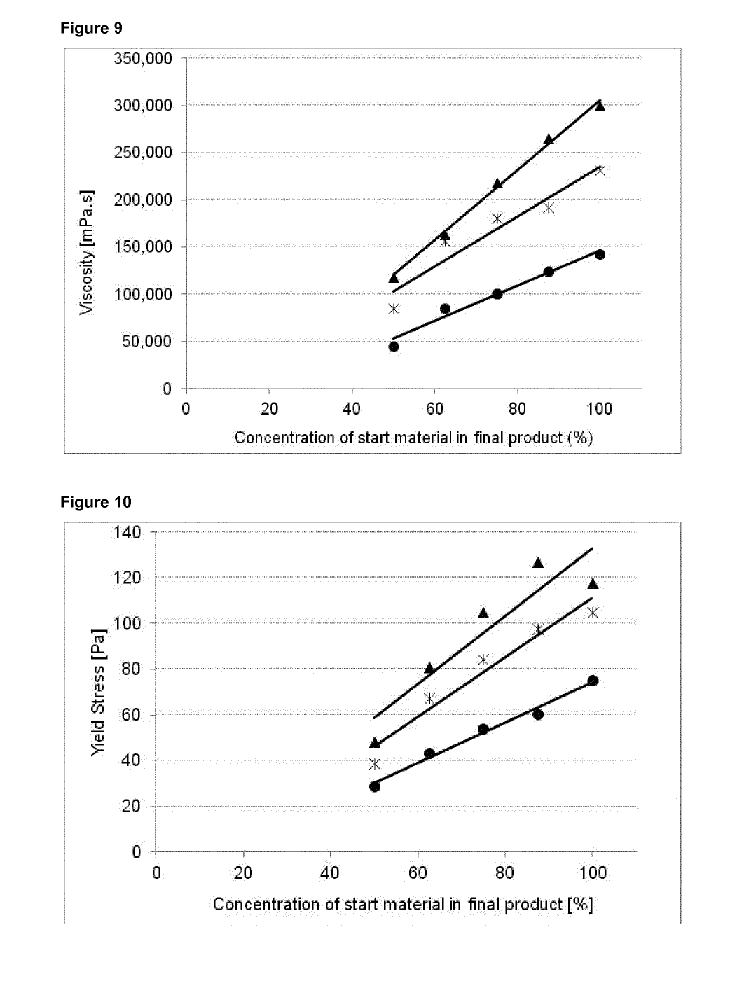

FIG. 9 plots the control samples (, not passed through the CDDM), and the samples that have been passed through the CDDM, operated in either rotating mode (▴) or static mode (*). The data points of the samples measured using the static CDDM are the average of two measurement series. It is shown here that the dynamic viscosity strongly increases when the samples have passed the CDDM. A concentration of actives of about 62-75% seems to give a similar dynamic viscosity as the control sample at 100% concentration. Whether the CDDM is operated rotating or static seems to make a difference. The viscosity of the compositions which were passed the rotating CDDM show a larger increase in viscosity than the samples which have passed the static CDDM. The viscosity linearly increases with the concentration of actives.

The yield stress of the various samples as function of the degree of dilution is plotted in FIG. 10. This figure plots the control samples (, not passed through the CDDM), and the samples that have been passed through the CDDM, operated in either rotating mode (▴) or static mode (*). The yield stress of the samples that have been passed through the CDDM is higher than of the control samples. A concentration of actives of about 62-75% seems to give a similar yield stress as the control sample at 100% concentration. Moreover, the rotation of the CDDM seems to influence the yield stress. The yield stress of the compositions which were passed the rotating CDDM show a higher increase in yield stress than the samples which have passed the static CDDM. For all samples holds that the yield stress seems to be linearly related to the concentration of the actives in the composition. The yield stress of the samples that passed the CDDM, increases with a higher rate as function of the concentration of actives, than the control sample.

This example shows that the viscosity and yield stress of the product which is mixed using CDDM is much higher than the product prepared in a conventional way, with the same concentration of actives. Or in other words, with a reduced concentration of actives, the same dynamic viscosity, viscosity profile as function of shear stress, and yield stress can be obtained as a product produced without mixing in the CDDM. In this case the concentration of actives can be decreased by about 25-38%, while keeping the same rheological properties.

1. A method for production of a structured liquid composition comprising water, a fatty compound having a melting point of at least 25° C. at a concentration of at least 1% by weight, and one or more non-ionic surfactants at a concentration of at least 1% by weight, comprising the step:
a) mixing the fatty compound in liquid form with a mixture containing the one or more non-ionic surfactants in liquid form and water, or
mixing the fatty compound in liquid form with the one or more non-ionic surfactants in liquid form, and mixing this mixture with water;
characterised in that in a next step
b) the mixture from step a) is introduced into a distributive and dispersive mixing apparatus of the Controlled Deformation Dynamic Mixer type,
wherein the mixer is suitable for inducing extensional flow in a liquid composition,
and wherein the mixer comprises closely spaced confronting surfaces at least one having a series of cavities therein in which the cavities on each surface are arranged such that, in use, the cross-sectional area for flow of the liquid successively increases and decreases by a factor of at least 5 through the apparatus.

2. A method according to claim 1, wherein the one or more non-ionic surfactants comprise a non-ionic surfactant having a HLB value ranging from 2 to 6.5, preferably from 4 to 6, and a non-ionic surfactant having a HLB value ranging from 6.5 to 18, preferably from 12 to 18.

3. A method according to claim 1 or 2, wherein the fatty compound is selected from one or more compounds from the group of fatty alcohols, triglyceride oils or fats, and mineral oils.

4. A method according to any of claims 1 to 3, wherein the Controlled Deformation Dynamic Mixer comprises two confronting surfaces (1, 2), spaced by a distance (7), wherein the first surface (1) contains at least three cavities (3), wherein at least one of the cavities has a depth (9) relative to the surface (1),
wherein the second surface (2) contains at least three cavities (4) wherein at least one of the cavities has a depth (10) relative to the surface (2),
wherein the cross-sectional area for flow of the liquid available during passage through the apparatus successively increases and decreases at least 3 times, and
wherein the surface (1) has a length (5) between two cavities, and
wherein the surface (2) has a length (6) between two cavities, and
wherein the surfaces (1, 2) are positioned such that the corresponding lengths (5, 6) overlap to create a slit having an offset distance (8) or do not overlap creating a offset distance (81), wherein the cavities are arranged such that the cross-sectional area for flow of the liquid available during passage through the apparatus successively increases in the cavities and decreases in the slits by a factor of at least 5 and
wherein the distance (7) between the two surfaces (1,2) is between 2 micrometer and 300 micrometer, and wherein
either the ratio between the offset distance (8) and the distance (7) between the two surfaces (1, 2) ranges from 0 to 250,
or wherein the ratio between the offset distance (81) and the distance (7) between the two surfaces (1, 2) ranges from 0 to 30.

5. A structured liquid composition, prepared according to the method of any of claims 1 to 4, comprising water, and
one or more fatty compounds having a melting point of at least 25° C. at a concentration ranging from 1% to 4% by weight, and
one or more non-ionic surfactants at a concentration ranging from 1% to 8% by weight, and wherein the total concentration of anionic surfactants, cationic surfactants, and zwitterionic surfactants is maximally 3% by weight,
and wherein the structured liquid has a dynamic viscosity of at least 80,000 mPa·s, preferably at least 100,000 mPa·s, measured using a Brookfield RV viscometer, fitted with a T-bar T-E spindle, at a rotational speed of 5 rpm, and a temperature of 25° C.

6. A structured liquid composition according to claim 5, wherein the concentration of fatty compounds ranges from 1% to 3.5% by weight, preferably from 1.5% to 3.5% by weight, and/or wherein the concentration of non-ionic surfactants ranges from 1% to 6% by weight, preferably from 1.5% to 4% by weight.

7. A structured liquid composition according to claim 5 or 6, wherein the one or more non-ionic surfactants comprise a non-ionic surfactant having a HLB value ranging from 2 to 6.5, preferably from 4 to 6, at a concentration ranging from 0.5% to 7%, preferably from 0.5% to 5% by weight, and/or
a non-ionic surfactant having a HLB value ranging from 6.5 to 18, preferably from 12 to 18, at a concentration ranging from 0.5% to 2%, preferably from 0.5% to 1.2% by weight.

8. A structured liquid composition, prepared according to the method of any of claims 1 to 4, comprising water, and
one or more fatty compounds having a melting point of at least 25° C. at a concentration ranging from 2% to 5% by weight, and
one or more non-ionic surfactants at a concentration ranging from 4% to 8% by weight, and wherein the total concentration of anionic surfactants, cationic surfactants, and zwitterionic surfactants is maximally 3% by weight,
and wherein the structured liquid has a dynamic viscosity of at least 60,000 mPa·s, preferably at least 80,000 mPa·s, measured using a Brookfield RV viscometer, fitted with a T-Bar T-D spindle at a rotational speed of 10 rpm, and a temperature of 25° C.

9. A structured liquid composition according to claim 8, wherein the concentration of fatty compounds ranges from 2% to 4.5% by weight, preferably from 2% to 4% by weight, and/or wherein the concentration of non-ionic surfactants ranges from 4% to 7% by weight.

10. A structured liquid composition according to claim 8 or 9, wherein the one or more non-ionic surfactants comprises a non-ionic surfactant having a HLB value ranging from 2 to 6.5, preferably from 4 to 6, at a concentration ranging from 3% to 7%, preferably from 3% to 6% by weight, and/or
a non-ionic surfactant having a HLB value ranging from 6.5 to 18, preferably from 12 to 18, at a concentration ranging from 0.5% to 3%, preferably from 1% to 2.5% by weight.

11. A structured liquid composition according to any of claims 5 to 10, wherein the total concentration of anionic surfactants, cationic surfactants, and zwitterionic surfactants is maximally 1% by weight, preferably maximally 0.5% by weight.

12. A structured liquid composition according to any of claims 5 to 11, wherein the concentration of polymers is maximally 2% by weight, preferably maximally 1% by weight.

13. A structured liquid composition according to any of claims 5 to 12, comprising an antiperspirant active, preferably comprising an aluminium compound and/or a zirconium compound.

14. A product for treating perspiration comprising a composition prepared according to the method of any of claims 1 to 4 and comprising an antiperspirant active, preferably comprising an aluminium compound and/or a zirconium compound, or according to claim 13,
and an applicator comprising a reservoir for holding the composition and a surface for applying the composition to the skin.

15. Use of a structured liquid, prepared according to the method of any of claims 1 to 4 and comprising an antiperspirant active, preferably comprising an aluminium compound and/or a zirconium compound, or according to claim 13 as deodorant or antiperspirant.
